(19) United States
(12) Patent Application Publication
(10) Pub. No.: US 2013/0253069 A1
(43) Pub. Date: Sep. 26, 2013

(54) Use of DMSO for Destroying Adipocytes and Eliminating Cellulite and Adiposities

(71) Applicant: Mariana Adélia Girotto Bussade, Sao Paulo (BR)

(72) Inventor: Mariana Adélia Girotto Bussade, Sao Paulo (BR)

(21) Appl. No.: 13/877,885

(22) PCT Filed: Sep. 16, 2011
(86) PCT No.: PCT/BR11/00367

§ 371 Date: Jun. 12, 2013

(30) Foreign Application Priority Data
Oct. 5, 2010 (BR) PI 1004030-7

(51) Int. Cl. A61K 31/10

(52) U.S. Cl. CPC A61K 31/10
USPC 514/708

(57) ABSTRACT

The invention relates to the use of DMSO for destroying adipocytes and eliminating cellulite and adiposities, more particularly to the concentrations used for destroying adipocytes (fat cells) and eliminating cellulite and adiposities, in both males and females, resulting in the destruction of adipocytes, or fat cells, and the elimination of fat cells, which, in turn, are affected by rancidity and contain toxins, that is, rancid, fat-containing cells generate cellulite since said cells sustain cellulitic nodules, which, in turn, are observed externally as the “orange peel” effect, which is more common in women and easily identified according to the degree of cellulite, and thus, by eliminating fat cells, adiposity is naturally reduced, and consequently, there is a decrease in localized body fat.

### TECHNICAL FIELD

The present invention particularly relates to the use of DMSO (Dimethyl Sulfoxide) in order to destroy adipocytes (fat cells) and eliminate cellulite and adiposities, in both males and females.

### BACKGROUND OF THE INVENTION

Most women, i.e. more than 99%, have or will have cellulite and, despite the fact this problem leads to a disease with low clinical implications, it brings great aesthetic concern, many times leading to constraint while using small clothes or bathing suits, being main consequences of cellulite of aesthetic order, with impairment of self-esteem and social interactions, especially when in advanced stages, not to mention the health problems inherent to it, of course.

The excess of localized fat present in several parts of the female and male body is also cause of anguish and, as well as cellulite, cause of a number of issues such as low self-esteem, dismay, depression and impaired social interactions.

According to World Health Organization (WHO), there are more than 1.1 billion overweight adults and 300 million obese adults.

Since it increases the risk of diabetes, arterial hypertension, heart diseases, joint diseases, psychiatric disorders, and certain forms of cancer, WHO considers obesity as one of the world's ten main threats to health integrity and one of the five main in industrialized countries.

According to studies from competent bodies, the number of obese people increases every day. For example, researches show there are about 17 million obese people in Brazil, which means 9.6% of the population.

The effects of the obesity along with modern life's stress, poor diet, sedentary life and lack of mental and physical balance can be devastating to human body.

According to recent researches, the adipocytes (fat cells) proliferate since birth until a certain age, more specifically until 20 years of age. People with normal weight accumulate 20-30 billion adipocytes, while obese people have 60-80 billion of such cells.

Every year, regardless one's weight, 10% of the fat cells die. However, they are replaced by younger cells with more need of fat.

After the twenties, the number of adipocytes is kept, they only swell or increase in size when the person grows fat, and shrivel or decrease in size if the person loses weight. When swollen, adipocytes are filled with fat and cause a number of consequences, such as obesity, the onset of the well-known cellulite and adiposities (localized fats), besides all heart and metabolism problems deriving thereof, of course.

This is the reason why it is so difficult to lose weight and keep shape and why the harmful “yo-yo effect” occurs, i.e., alternating weight gain and loss resulting from consecutive diets to lose weight.

There is fat inside an adipocyte. When a person is in a calorie-restricted diet, the cell size is reduced, but that cell is not eliminated. Therefore “weight loss” occurs.

Fat cells constantly diminished effectively result in weight loss and reduction of body measures. However, once calories are back those cells quickly grows back to same size or even larger. That is, a person can become even fatter than before starting the diet.

Several signals and symptoms have been related to what is referred to as “cellulite”, which makes difficult for the understanding and therapeutically guidance regarding the pathology, especially for those with no skills in the art: thus, it is described as accumulation of fat tissue (fat cell+cell rancidity) in certain sites (being confused with lipodystrophy) or even as an exclusively dermal process.

For those with no skills in the art, cellulite belongs to the field of localized obesity, with an aspect of adipose hypertrophy characteristically edematous, followed by well-known alterations to skin appearance (“orange peel skin”), notably on hips and thighs.

Hardly dissociated from obesity, to which is often connected, cellulite features more complex pathogenic issues, and the term should be saved for superficial infiltrations, either localized or generalized. Whatever are the ways cellulite is developed, it looks like some initiating factors interfere by means of a circulatory vasomotor disturbance in connective tissues which entraps a critical vasculature and sensitive innervations at the same time.

Briefly, cellulite is the accumulation of fluid, fat and water in tissues, forming localized bags attached to a net of adherence and sclerotic tissues that would cause changes to local blood circulation and tissue oxygenation, thus forming the “orange peel skin” appearance.

Cellulite is accepted as an autonomous anatomic and clinical entity, different from obesity or lipodystrophy; however, it is commonly associated to both.

With peculiar clinical and etiopathogenic characteristics, cellulite often requires a wider approach, involving psychological, biological, and sociological aspects.

Psychological and social concerns around cellulite are complex and hard to approach; there are no purely psychosomatic implications and patient's complaints are not supposed to be taken as mere aesthetic concerns, since it often reflects an enormous anxiety.

There is this permanent and insistent pressure to fit in the utopic representation depicted on the cover of female magazines: the lean-shaped figure who wants and gets all men; powerful, free, and perfect.

Fashion is constrained to beauty. Its language turns women's bodies into objects that consume and are consumed.

As long as the psychological and social concerns around cellulite are discussed, its complexity and difficult theoretical approach must not be ignored.

More than thirty pharmacological and therapeutic properties have been found for DMSO, which result from its ability to interact or combine with nucleic acids, carbohydrates, lipids, proteins and many drugs without irreversibly changing the molecular configuration. These properties assure its acknowledgment as one of the most versatile drugs ever known.

On the other hand, since its introduction as medicine, DMSO has generated controversy in the scientific environment, dividing opinions, since its evaluation is extremely difficult due to the numerous and complex variables involved, especially when blended to other actives, since DMSO is a powerful active diffuser.

DMSO is one of the most powerful antioxidants or inhibitors of hydroxyl free radicals, with the advantage of being usable topically, orally and/or via parenteral, with almost no side effects. Priority (known) intended uses of DMSO are related to musculoskeletal pathologies, of degenerative, inflammatory, and/or traumatic nature.

Among the intended uses and properties described in literature, it could be mentioned the topical application for musculoskeletal diseases; topical inflammatory agent; topical pain reliever; itch reliever; skin problems; vasodilating effect; rheumatoid arthritis; penetrating and diffusing effect; carrier of other drugs, potentializing effect of other substances; herpes zoster; slight bacteriostatic agent; macular and papular amyloidosis; scleroderma; improves blood circulation to all tissues; muscle relaxant; immunomodulatory action; increases cell function and differentiation; cryoprotective action; calming effect.

The application is systemically administered for rheumatic diseases, gastrointestinal disorders; cerebral edema(traumatic); diuretic; interstitial cystitis; vasodilating effect; urinary problems; lung problems; action on lipid metabolism; lung adenocarcinoma; chronic prostatitis; schizophrenia; Alzheimer's disease; antagonism of platelet aggregation; protection against ischemic injury.

For all these reasons, the Applicant, being active in the market and always concerned with the evolutions of his/her products, with the objective of always offering the best for the consuming public, has already filed with INPI [Brazilian Institute of Industry Property], Patent Application no. PI0504655-6, filed on Oct. 17^{th}, 2005, entitled “DMSO BASED FORMULATIONS FOR USE IN FIGHTING CELLULITE AND OTHER ADIPOSITIES”, which describes the use of DMSO-based formulations to locally fight body adiposities, these being: paniculopathy edemofibrosclerosis, localized fats; flabbiness; and fat in general; such formulations use different percentages of DMSO in different solutions, these being: gel, cream, aqueous solution and solution for mesotherapy.

Said patent no. PI0504655-6 is based in using DMSO to fight cellulite and other adiposities, i.e., since cellulite is mainly characterized by the presence of rancid, ill-nourished, poorly oxygenated fat, with accumulation of toxins, besides other factors contributing to its formation, the researches made to reach to a definitive solution for the problem come upon not only fighting the cellulite, where there is only an improvement from the “orange peel skin” appearance, but in eliminating the problem, i.e., it is not about palliatives anymore, but a definitive solution for the problem.

It is correct to say that, regarding the destruction of adipocytes, which are also present in cellulite, it is also considered using DMSO to eliminate adiposities (localized fats), since destroying fat cells naturally eliminates adiposities.

By destroying an approximate amount ranging from fifty thousand (50,000) to five-hundred thousand (500,000) adipocytes (fat cells) per delivery of DMSO, fat cells (adipocytes) are effectively destroyed. The “rancid” fat cells (adipocytes) generate the cellulitic nodule. Eliminating the adipocyte (fat cell) will eliminate cellulite and consequently the adiposity.

Many denominations have been proposed over the years for cellulite, such as panniculitis, panniculosis, lipedema, mesenchimatosis, fibro edema geloid, panniculopathy, etc.

Despite of the diversified nomenclature, all authors agree in several essential, constant aspects in cellulite, that is, it affects most women; it is localized in very specific areas; it is frequently associated to lipodystrophy or obesity; it is caused by a hydric unbalance due to the polymerization of mucopolysaccharides; interrupted or hindered intercellular diffusion; changes in microcirculation.

Cellulite, despite of the inadequate term, constitutes an autonomous anatomic and clinic entity.

It is a dysmorphia related to a dystrophy localized in cutaneous connective tissue and subcutaneous adipose tissue, which hypertrophies. Both parameters, cutaneous and adipose, are affected at various levels.

In young patients, cellulite is limited to an adipodystrophy until a big change with loss of elasticity and dermal sclerotic involution. It is actually an accelerated and local skin aging process. So, certain characteristics contrast and set its identity, be it its exclusive incidence in women; its topography, gynoid type, located in hips, lower limbs and, in minor degrees, in abdomen subumbilical area; its occurrence is more frequent during puberty or in a moment of sex life; sometimes, as consequence of a massive, long-lasting hormone therapy with estrogens or corticoids. The role of the birth control pills has been reminded by some authors due to its refractivity to diets and drugs, generally active in treatment of common obesity; certain changes in incidence, more or less frequent connection with premenstrual syndrome, cyclic edema syndrome, deficient venous return in lower limbs, and deficit of certain muscular groups, specially gluteal and pelvis-trochanteric muscles.

The main function of the fat tissue is metabolic. It acts as an energy regulator of animal organisms, adapting the stock of lipids and carbohydrates under the form of triglycerides, at the expense of energetic spending.

The main characteristic of the fat cell is storing fat when the body is fed and returning it on modulated mode, under the form of free fatty acids, when necessary. Briefly, the fat tissue is like an energy bank; the deposits are made when the body is fed and the debt orders are transmitted at all times and withdrawn by hydrolyzing fat into free fatty acids. If, during a sufficiently extended time, the amount of deposits surpasses the amount of withdrawals, obesity occurs.

Besides its mechanical role of support tissue and lining for several organs, the connective tissue has critical importance for nutrition and their functioning; ultimately, it controls physiology. The connective tissue is formed, in various proportions, by cells (fibroblasts) and fibers (collagens, elastin, and reticulin) included in a base substance partly fluid, partly solid.

An intermediate between vessels and other tissues (glands, muscles, etc.), the connective tissue is where exchanges within organism occur. By means of its free cells and, specially, the leukocytes crossing it, it has an essential role in defending organism which, on the other hand, guarantees the material necessary for its support, through changes to base substance, which becomes hard, like bones and cartilages.

Whatever is its aspect, morphologic type, or histologic manifestations, the connective tissues always include three elements, which are cells; fibrillar formations, and a base substance.

These three elements are always found in all types of connective tissue, in diverse ratios, as diverse as their aspects are.

Regarding the predisposing factors for cellulite, the sexual factor is worth mention, i.e., sexual hormones (estrogens, progesterone and androgens) guides the topographic distribution of fat tissues, both in men and women, and impart a special characteristic to this tissue constitution.

It is during puberty when secondary sexual characteristics appear, with the formation and distribution of fat tissue. Estrogens and progesterone induce the growth of adipocytes and in larger number in some parts.

Hormones are also another important factor, since the female organism is more and more taken by significant amounts of estrogens, there are changes to certain area due to the activation of hormonal receptors for estradiol and progesterone, forming molecular chains responsible for changes in interstitial metabolism in the skin and the subcutaneous tissue.

Several factors trigger cellulite, among which is the emotional factor, since some authors impart a predominantly psychosomatic character to cellulite after connecting its onset and development to psychological and neurovegetative factors.

For example, a poor regulation in estrogen production, as consequent hydric retention and metabolic alterations, can be mentioned as point of start for the onset of the pathology.

Another aspect is diet, where fat excess associated to the site of cellulite onset certainly worsens the picture, the reason why a diet with increased or large ingestion of fat and carbohydrates or even low consumption of water and excessive consumption of salt aggravate the microcirculatory picture with increased capillary resistance.

There are also toxic elements, where the isolated or concomitant consumption of coffee, cigarettes and anovulatory drugs largely promotes the onset or aggravation of cellulite, mainly due to changes in microcirculation.

Sedentary habits, where the lack of regular physical activities causes diminished loco-regional circulation, with reduced calorie spending, reduced adrenergic stimulation in adipocyte receptors, diminished use of glucose by muscles, increased muscle and sinew hypotony, muscular atrophy, and increased fatty mass.

Posture habits are also important, since the preferential position during the day, like the habit of sitting down can aggravate cellulite by compressing ganglionic chains of the popliteal and inguino-crural areas, due to the resistance offered to the circulation of venous and lymphatic return, maintaining the loco-regional edema.

Posture and orthopedic alterations are always present as aggravating factors, providing diminished lymphatic circulation, especially in cases of lordosis and hyperlordosis.

The lack of pumping in “plantar heart”, in case of flat feet or use of shoes with inadequate insoles or plant support vices, promotes a deficit of venous return, thus forming the edema.

Internal compression: lack of guidance regarding clothing or fashion trends, besides tight clothes (causing compression of the superficial dermal plexus for a long period of time) contributes for the onset of edema in the whole compressed area and aggravation and onset of cellulite.

Pregnancy actuates as a mechanic component, impairing the venous return of lower limbs. Increased levels of estrogen and progesterone promote diminished tonicity in vascular walls, favoring its dilation with consequent changes to vascular permeability.

Circulatory problems: insufficient venous return and varicose veins aggravate microcirculation blockage, also favoring the overflow of fluids to interstice, thus worsening the edema, with consequent compression of microcirculation, thus aggravating cellulite.

Antihistaminic drugs, antiserotonins, antithyroid agents, betablockers, corticoids and antidepressants also contribute to onset or worsening cellulite.

Usually, cellulite is present in women, being located, by order of incidence frequency, in medial and posterior thighs; gluteal region; anterior and lateral thighs; internal knees; abdomen; upper limbs (arms); deltoid region.

To identify cellulite, put your hands over the skin with probably onset of cellulite. Press the area with both thumbs and both forefingers concomitantly extended. If the skin has a rugged aspect, like an orange peel, there is cellulite.

If this test is positive, the onset of cellulite is proven and no other evidences are necessary. In more advanced stages, this test becomes painful.

Another form to identify cellulite is by pressing the skin, and lifting it, which is followed by a strong pain. This is also a case of cellulite.

If the skin lifts when slightly pressed (like one should press a tomato), and it is extremely painful when pinched, there is cellulite.

There are development stages of cellulite, starting by mild cellulite, which is only noticed by the skin test. When the person is in a horizontal position, the skin shows no alterations. Only by standing up and pressing the skin with both hands one can notice the “orange peel skin”, or “mattress phenomenon” typical of cellulite.

The second stage is the medium cellulite, which appears mainly in thighs and is visible without tests, when the person is lying down. When there is lateral light, the skin marks are easily spotted. “Pinching” test is always positive.

The third and last is the severe cellulite, which is visible both standing up and lying down, since the skin is rugged and flabby, like a “nut sack”. Connective tissue fibers are almost totally damaged, being currently referred to as Level 4.

The microcirculatory unit, angion, qualified by cellular life rotatory plate, is the center of tissue balance and, therefore, different vascular systems must adapt to circulatory variations.

When the compensation mechanisms of venous disturbances are surpassed, vascular and tissue structures are changed.

The microcirculatory system is terminal and intensely branched in superficial dermal plan, located in the papillary derma. In deep, they are located around fat cells.

Such microcirculation shows an interesting hemodynamic balance, regulated by gradients of hydrostatic and osmotic pressure.

Hydrostatic pressure promotes the escape of fluids through the vascular wall to the interstitial medium and has its return in vascular level by means of the oncotic pressure.

When the balance point is changed between gradients of hydrostatic and osmotic pressure, a stasis is produced, with changes to the microcirculation which, along with other factors, contribute to onset or aggravation of cellulite.

The fat dermal tissue with cellulite, where the disturbances leading to cellulite are located, is complex. Electronic and biochemical microscopy allow distinguishing the main elements, i.e., the connective cells, notably the fibroblasts, where the components of the connective tissue are elaborated; cells with diverse functions: macrophages, mast cells, plasmocytes, etc., collagen fibers of protein nature, among composing amino acids, alamin, proline, hydroxyproline and glicocola are determinant; the elastic fibers: due to its protein nature, they can extend and bear strong tractions, and go back easily to the initial position; the reticular fibers: are fibers anastomosed with each other, thus forming a structure like a net.

There is the base substance: is the medium where the cells and fibers of the connective tissue are.

The fat cells or adipocytes: are concentrated in the deeper layer of the derma (hypoderm) and are found on a thin network of collagen fibrils. The adipocytes are grouped in the shape of “grape bunches”, called fatty lobules, separated by connective tissue walls or lobular septa. The connective tissue é richly vascularized, since the exchanges between fat cells and blood flow are intense.

The regular connective adipose tissue comprises cells: fibroblasts, macrophages, mast cells, plasmocytes, etc.; collagen fibers, grouped in beams; thinner elastic fibers providing elasticity to tissues; reticular fibers forming a net; the base substance in which cells and fibers are in; the fat cells, or adipocytes, are concentrated in the deeper layer of the derma and grouped in “mounts” (fatty lobules), separated by connective walls, where blood vessels and nerve endings pass through.

The cellulitic tissue shows an important structural modification, which is: local fat cells increase in number and volume; besides, thickening and proliferation of interadipocyte and interlobular collagen fibers are noticed, causing tissue engorgement; drainage circulation is considerably reduced, and the fibroblasts are incarcerated; elastic fibers become fragile and tear apart; sclerotic fibers press vessels and nerves like a “ruff”.

It becomes, therefore, a poorly oxygenated, poorly fed, unorganized tissue, without elasticity. It is painful due to the pressure made to nerve roots (painful to touch, pressure, and palpation).

Cellulite must be promptly differed from obesity. Even though both problems are often connected, cellulite might affect thin individuals.

Like any disease, cellulite has several phases. Lageze was the first to describe the anatomopathology of cellulitic infiltrations. Four phases were described, in which the first brief phase is not noticed by the patients. It is a purely circulatory phase, which essentially comprises a venous and lymphatic stasis. In its normal state, the ability of emptying veins corresponds to the blood volume provided by the arteriole. The capillary sphincter lets the blood flow slowly, and this slowness allows excellent conditions for tissue oxygenation, as well as good efficacy in removing tissue waste. This balance might be disturbed by several circumstances: emotional issues, heat, hormonal disturbances (progesterone, which relaxes vein tonicity and stops return circulation), thyroid disorders and also local compression (tight boots, jeans, girdles, panty hoses) and even the growth of fat cells.

All these disturbances will cause diminished oxygenation and local asphyxiation. These combined structures suffer and then the second phase begins.

The second phase is exudative. The arteriole capillary vasodilatation caused by the stasis is accentuated and the subcutaneous cellular tissue is invaded by an exudate of vascular origin, containing mucopolysaccharides and electrolytes, which dissociate connective fibers and changes local nervous terminations. No leukocytary influx, no local eosinophilia can be evidenced, the electrophoretic analysis of the tissue fluid shows that its concentration in proteins is inferior to the blood, and thus shows its static and non-inflammatory nature.

In the third phase, fibroblasts appear, setting a fibrous scaffold which progressively turns into collagen. This is the phase of the cellulitic nodules.

The fourth and last phase has scarring, atrophic and irreversible. There is sclerotic retraction. The arterioles are taken by endarteritis and periarteritis, and the nerves are smashed by a set of inexorable fibrosis.

Very frequently, this process is followed by a hypertrophic development of the hypoderm fat tissue. The fat lobules, being too bulky for the rigid and inextensible fibrous scaffold containing them, take a very firm consistency, due to increased pressure. Therefore, the nerves and vessels in the connective wall are under pressure.

Described by Coperman and Mason, an aqueous retention in fat cells occurs, which would be directly dependent on glucocorticoids.

The development stages of cellulite under electronic microscope occur in the hypertrophy of fat cells, where there is a pathologic accumulation of lipids developed in the adipocyte, causing hypertrophy of the fat cells. The fat cell core is pushed towards the girth by the lipid vacuoles.

The thickened interlobular septae are an aggravation of the cellulitic state and are characterized by a proliferation of the collagen fibers. They compress the vessels and nervous terminations, then cellular exchanges diminish, and circulatory obstructions occur, as well as pain.

The hypertrophy of the collagen fiber network among fat cells causes an alteration to the exchanges between bloodstream and adipocytes. Therefore, lipolysis enzymes cannot reach the adipocytes.

Venous stasis imparts an increase in capillary pressure. The capillary permeability increases, which turns into an evasion of fluids and proteins of high molecular weight for the interstitial connective tissue.

The increased permeability and interstitial inundation cause a lymphatic overcharge with onset of the edema.

The release of aggressive substances, like histamine, serotonin, and prostaglandins trigger a series of tissue reactions.

If the excessive protein is not depolymerized by macrophages, a fibroblastic stimulation and onset of fibrosis occur, which maintain and aggravate the venous capillary lymphatic stasis, thus closing the pathologic vicious circle.

The cutaneous and adipose parameters are affect in several levels, which makes possible to divide the evolution of cellulite into four stages:

1st STAGE—Congestion phase—polymerization of the base substance; difficult venous return; alteration of capillary permeability; periadipose interstitial edema; lipedema with adipocyte hypertrophy.

The first consequence of the venocapillary stagnation is the widely increased endothelial permeability with consequent flooding of the tissue.

2^{nd} STAGE—Exudation phase—infiltration: adipocyte atrophy; adipocyte degeneration; hypertrophy and hyperplasia of reticular fibers; cutaneous and hypodermal microangiopathy.

The stasis persists. The hemodynamic balance is undone, with leakage of plasma, electrolytes and mucopolysaccharides.

The edema compresses connective fibers and cutaneous elasticity diminishes.

3^{rd} STAGE—Fibrous reorganization phase: adipocyte dissociation and rarefaction; formation of micro nodules; loss of dermo-hypodermic boundaries; onset of sclerosis in dermal connective tissue; focal hyperkeratosis; venocapillary insufficiency; microaneurysms; sclerosis of the artery-arteriole average; diffuse microbleeds.

Throughout some weeks or months, the connective fibers become more turgid, with increase in amount of mucopolysaccharides from 6% to 7%.

The deterioration of collagen is more evident, forming amorphous and disorganized blocks, completely unbalancing its structure.

Connective trabeculae incarcerate the adipocytes, full of triglycerides and water, causing the “orange peel skin” appearance, hindering intercellular and vascular exchanges even more.

The formation of capsules of sclerotic collagen fibrils begins, involving colonies of adipocytes (around 100), thus originating micronodules.

4^{th} STAGE—Healing or sclerosis phase—it is the definitive phase of cellulitic process and hardly reversible.

Blood supply is diminished, causing important nutritional changes to connective tissue.

Newly formed collagen fibers thicken, notably characterizing the sclerotic process: presence of macronodules; diffuse lipo sclerosis; dystrophy and atrophy of epidermis and annexes; hyaline degeneration of derma; capillary hypovolemy; microvaricose veins; microcirculatory deficits.

The fusion of several micronodules with microscopic dimensions causes the macronodules, perceptible to palpation. (PSN=Painful subcutaneous nodule).

DMSO (Dimethyl Sulfoxide) is an organic chemical compound derived from wood pulp. It is a byproduct of the timber industry and it is also produced through the normal decomposition of plants.

DMSO has been used as a commercial solvent since 1953 and it is one of the most studied pharmaceutical agents of our time, although the least understood. More than 40,000 articles about its chemical properties were published in scientific journals that, along with thousands of laboratorial trials, offer strong evidence of a wide variety of properties. Globally, more than 11,000 articles were written about its medical and clinical implications, and in 125 countries around the world, including Canada, England, German, and Japan, physicians prescribe DMSO for several diseases, including pain, inflammation, escleroderma, interstitial cystitis, arthritis, etc., however, none of these articles have citation regards its use for destroying adipocytes (fat cells), eliminating cellulite and adiposities.

DMSO is a substance of formula C₂H₆SO, molecular weight 78.13 and freezing temperature of 18.5° C. (65.3° F.).

DMSO is in liquid form, is colorless and has a slight odor. The specific temperatures or temperature ranges in which physical state changes occur are below:

Melting temperature: 18.5° C.; boiling point: 189° C.; ignition temperature: 300-302° C.; flash point: 95° C.; explosive limit: 1.8-63.0%.

DMSO is a molecule with an atom of sulfur in the center, bonded to two methyl groups, an atom of oxygen and an unbounded electron, which is bonded in tetrahedron-shaped points, as below:

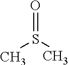

The elevated hygroscopic capacity stems from its intense hydrogen affinity, forming stronger bridges than those formed between water molecules.

This causes pure DMSO to move quickly to concentration between 66-67% if exposed to the environment, reason why it must be kept in a hermetically closed container. An exothermic reaction is noticed when topically administered DMSO reacts with water from air and tissues. This chemical peculiarity is related to several properties of the drug and its solvent capacity, particularly on acrylic and polyurethane, thus requiring special care so as to not react with bandages, paraments, and equipment made of such materials.

The crystalline structure of DMSO indicates the presence of a weak hydrogen bond, however, in fluid state, assume a structure of chains aligned by sulfide and oxygen poles.

It is sufficient to say that DMSO is an extraordinary solubilizing agent, dissolving diverse chemical substances with its hydrophilic and lipophilic properties. DMSO is freely miscible in polar solvents as water and alcohol, as well as it is relatively miscible in non-polar solvents such as chloroform and methylene chloride.

The drug has the ability of chemically by heating, thus having a great thermogenic effect, releasing sixty calories per gram of DMSO when mixed with water.

The purest concentration of DMSO that exists is 99.5%, which can be determined by putting it in a freezer for two hours, the time when the fluid becomes solid as ice. DMSO only does not freeze when diluted in 50% water.

DMSO is a molecule soluble in aqueous medium and organic medium. It is a very efficient solvent for water-insoluble components and is a biologically and therapeutically active substance.

Dimethyl sulphona and dimethyl sulphate are the main degradation products.

It is mainly excreted via urine; however, due to the odor, the air seems to be the main excretory way, but it responds for less than 3%.

The absorption, excretion, and metabolism of the dimethyl sulfoxide were studied in humans by gas chromatography and radiometric techniques. The drug was promptly absorbed when dermally administered, where the peak of plasmatic levels occurred after 4-8 hours. The drug orally administered was quickly absorbed, reaching the plasma peak within 4 hours. Serum levels of DMSO were undetectable after 120 hours.

Dimethyl sulphona (DMSO2) appeared in plasma after 48 hours and persisted in plasma for approximately 400 hours. Urinary excretion of DMSO after oral and dermal administration was equal to approximately 13% and 30-68% of the dose, respectively. Excretion of DMSO₂ was approximately 5-10% and 21-23%, respectively.

After dermal delivery of a DMSO solution at 70%, plasmatic levels of DMSO were maximum after 4-8 hours and then decreased with half-life of approximately 11-14 hours, until after 36-48 hours, when DMSO could not be detected any longer. However, plasmatic levels of DMSO₂ were not maximum until after approximately 60-70 hours. After 312 hours, levels were low but still detectable.

Urinary excretion of DMSO started a little after drug administration and continued for 48 hours. After that, very low DMSO was excreted. Total excretion of DMSO reached an average of 13% dose. Urinary excretion of DMSO₂ became more significant in approximately 8 hours and continued for 456 hours, total average of amount excreted was equal to 17.8% dose of DMSO. Therefore, in this experiment, an average of 30.8% dose was reported by urinary excretion of DMSO and DMSO₂.

The results presented showed that DMSO administered either dermally or orally to humans is excreted partly as the drug without modification and partly as the metabolite DMSO₂.

Current findings show that DMSO was quickly absorbed even when dermally applied to human beings or orally administered. The fact that the plasmatic levels of DMSO are lower after dermal administration than after oral administration suggests the possibility of the dermal absorption being less complete than absorption via gastrointestinal tract, which is also suggested by the low recovery of the drug when dermally administered. 1 g/kg of DMSO was dermally administered to 10 additional subjects in fluid form and in gel form (results not published by the authors) to another 10 subjects, and plasmatic levels and the urinary recovery of drug was even lower (less than 10% of the dose was recovered in urine). It is possible that some loss of DMSO occurred due to evaporation through skin surface.

The excretion of DMSO and/or metabolites in the air expired in human subjects in dermally administered DMSO was also studied (results not published by the authors) and reported for only a smaller fraction of the dose. Kolb et al. (1965) estimated that approximately 3% of the dose was excreted in the air dimethyl sulfide.

There is not a totally safe product, be it water or a tablet of salt.

However, DMSO is a safe and well tolerated drug. After analyzing the results of numerous studies and reports, it was observed that DMSO has an excellent safety and tolerability profile when administered orally, topically, intradermally, even via endovenous administration.

Unlike what was initially published in 1960s, DMSO toxicity is low. Even with the side effects, when there is inadequate indication, concentration, dosage and administration readiness, no anemia or kidney failure were observed.

Its topical application, although well tolerated, can cause a slight and transitory sensation of burn in the application site, flushing, skin rashes, itch, erythema, scales, dermatitis, characteristic odor in the body (an odor similar to garlic can last up to 72 hours due to the prolonged presence of dimethyl sulfide, a dimethylsulfoxide metabolite), occasional blisters, dry skin, pustules and pain in the application site. A heat reaction can occur if administered in humid skin. Concentrated solutions of DMSO release heat when diluted in water, and it has been suggested that such exothermic reaction causes the degranulation of mast cells and damage to the small veins of the skin. At the same time, DMSO undoubtedly has local osmotic effects directly in dermal cells.

These adverse reactions result from the vasodilating action of DMSO and are increased with prolonged use, in high concentrations or by occlusive administration. Skin reactions are the most common adverse effects after topical application of dimethyl sulfoxide. These reactions appear generally with the use of high concentrations of the active principle and are generally reversible after drug discontinuation. Some gastrointestinal effects were reported, like nausea and vomit; loss of appetite; diarrhea and constipation; disturbance of taste and bad breath (breath and taste similar to garlic occur to almost all subjects using dimethylsulfoxide).

Probably due to the effect of histamine release, topical use of dimethylsulfoxide can cause eosinophilia as reported in some subjects receiving DMSO at 80% gram/kilogram per day topically for 14 days and 90 days.

Hypersensitivity reactions can occur after topical administration of dimethylsulfoxide. Rash and even blisters can be noticed probably due to the release of histamine with the use of DMSO. It is also suspect of causing immunosuppressive action.

Topical delivery (gram/kg per day) of DMSO 80% applied to 60 subjects for 14 days caused sedation 52%, headache 42% and dizziness 18%. Less than 5% of these subjects reported the occurrence of symptoms of dry nasal passages, dry or painful throat and cough. Peripheral neuropathy was reported in some cases of topical use of DMSO.

No ocular adverse effect was observed in subjects receiving topical solution of dimethylsulfoxide. Studies in animals documented changes to lenses, however, some intensive studies and clinical experience failed to document any similar effect in humans.

In an international symposium carried out in New York City, sponsored by The New York Academy of Sciences, on Jan. 9^{th}, 10th, and 11^{th}, 1974, 82 papers were presented, and almost all of them shared the opinion that DMSO is effective in treatment of diseases and nearly non-toxic.

DMSO is in Pregnancy Category C, according to FDA (no studies in animals disclosed adverse effects to fetus and there are no controlled studies in women or studies in both women and animals available. Drugs must only be administered if the beneficial potential justifies the potential risk to the fetus).

It is unknown if DMSO crosses the placenta. Teratogenic effects were observed in animals exposed to dimethylsulfoxide.

There are no suitable and well-controlled studies in pregnant women. Dimethylsulfoxide must only be administered during pregnancy if the beneficial potential justifies the potential risk to the fetus. Thomson Lactation Rating:

The risk to the baby cannot be discarded. Available evidences and/or expert consenso are inconclusive or unsuitable to determine the risk to the baby when administered during nursing. Comparing beneficial potential of the treatment against potential risks before prescribing such drug during breast-feeding is recommended.

No report describing the use of dimethylsulfoxide during breast-feeding is available, and the effects in the infant exposed to the drug in milk are unknown. It is not known if DMSO affects the supply and composition of breast milk. Until there is more data available, care should be taken when considering the use of dimethylsulfoxide in nursing women. There are no reports describing the use of DMSO during the human lactation or measuring the amount of the drug excreted in milk, if any.

DMSO is a separator of hydrogen limit molecules, agent of cell differentiation, scanner of hydroxyl radicals, intercellular electric uncoupler, mobilizing agent of intracellular lipoprotein derived from low density cholesterol and cryoprotectant.

One can define DMSO according to the molecule characteristics, being: small, simple and with important biological and physical properties; has exothermic reaction properties (when diluted in water, produces heat); combines with hydroxyl radical by adding water and eliminates the complex via kidneys; replaces water inside the cells, helping controlling intracellular free radicals to maintain cellular homeostasis; increases cellular membrane permeability, allowing cells to release toxins; increases saturation of tissue oxygen through local vasodilation mechanisms by expanding blood vessels, diminishes platelet aggregation and increases oxygen by additional absorption through blood vessels; tends to improve blood supply through dilatation of small vessels and improves microcirculation, especially in upper limbs, being used in subjects with diabetic neuropathies and even as a preparation for surgeries, when the surgeon wishes to increase the blood flow to that specific part of the diabetic leg that eventually has surgical indication to replace circulation or to be amputated so as to preserve the remaining limb; one of the rare substances with antioxidant power over hydroxyl radical is DMSO, which forms a dimethyl sulphona associated to water to which, along with hydroxyl radical, is excreted. Hydroxyl neutralization, which is the probable cause of degenerative inflammatory processes, will determine control over rheumatic processes; free radicals are complex chemical components that might alter the metabolic function of all organic systems and are produced by the air we breathe, radiation, pollution, etc., which form superoxides, peroxides and also the hydroxyl radical, which determine cell lesions and degenerative e diseases. DMSO interferes with the production of free radicals, developing cellular homeostasis.

DMSO further shows the following cellular and molecular effects: inflammatory process; lipid metabolism; cellular cycle; protein expression; cell differentiation; molecule bonding; enzymatic activity; Scanner of reactive species of oxygen; cell polarization; and cryopreservation.

DMSO penetrates into the cellular membrane and causes an increase in osmolality both inside and outside the cell, thus preventing significant hemolysis due to the formation of an osmotic gradient.

Results show that DMSO penetrates even deeper inside the double lipid layer than water. DMSO increases the permeability of the stratum corneum and accentuates the penetration of other chemical substances through skin.

The possible clinical use of DMSO as vehicle for the transcutaneous transport of other drugs has been thoroughly studied. DMSO can be an optimal vehicle for the quick transcutaneous penetration of certain drugs intended for localized effects.

DMSO strongly interacts with the membrane surface, probably dislocating water and changing the structure of the double lipid layer.

It was specifically shown that DMSO has influence on the interactions of the lipid membrane differently from other solvents. The interaction of DMSO and biological membranes can have an important influence on the membrane.

The interaction between DMSO and the polar membrane surface affects the closure, enclosing the chain of hydrocarbons of lipid molecules. This apparently increases the defects in the structure of the double lipid layer, which must result in increased permeability. The biological and therapeutic impact of DMSO results in its ability to serve as vehicle, carrying drugs to inside the cell due to the fact that DMSO increases membrane permeability.

DMSO is characterized by showing the following action mechanisms: formation of biologic mediators; formation of cell immunomodulators with action at angion level, organ of internal secretion, center of tissue balance and microcirculatory unity; regulates the circulatory system, with increased cell immunity; has action in level of enzymatic systems, with formation and acceleration of intracellular lipolysis; increased thermogenesis and basal metabolism, with increased heat and energetic spending; has detoxifying, venotrophic, lymphokinectic, lipolytic, and enzymatic action; it is orthomolecular, hormonal and detoxifying.

Formulations containing DMSO actuate as follows: accelerate metabolic, combustive and oxidation processes; increase membrane transportation, eliminating toxins with cellular recovery and reactivation; increase vascular permeability; improve microcirculation, with improvement of cellular interchange; increase local oxygen captation, facilitating cell restoring and cell metabolic normalization; increase formation of biological cell mediators and immunomodulators, such as prostaglandins, interleukins, endothelins, cytokines, proanthocyanidins, etc., with modulation and selective distribution of energy with restoration of the biologic, biochemical and energetic processes; has action anti-free radicals and acid basic balance; promotes the tissue reorganization with more organic interaction; has emulsifying action, that is, has the ability of penetrating the adipocyte, acting as emulsifying agent, changing the physical and chemical characteristics of the stored intracellular lipids. Thus, they would be removed from intracellular space, being release in blood and lymphatic streams, and then eliminated through kidneys; promotes the release of free fatty acids more quickly, through biochemical mediators “inducing an unidirectional bomb” by supplying respiratory chains of the mitochondria, so the adipocytes have their excessive fat removed; stimulate enzymatic systems of the cellular membrane, with inhibition of phosphodiesterase and activation of adenyl cyclase, thus activating the lipolysis system of the organism, transforming fat through the hydrolysis of triglycerides in glycerol into fatty acids; increase muscular toning with due increase in formation of ATP, amino acids and proteins.

DMSO activates acid sphingomyelinase and accelerates LDL (low density lipoprotein) intracellular mobilization; increases the transference of non-esterified cholesterol among membranes; prevents expected increase of serum cholesterol in 50%, but does not prevent the accumulation of cholesterol in aorta tissues, when administered in cats with a glass of water along with a cholesterol-based diet; reverts the abnormal LDL process in the mutant fibroblast Niemann-Pick disease, characterized by excessive LDL accumulation in lysosomes and by delayed induction of cell homeostasis response associated to the captation of LDL by mutant cells. Accelerates the intracellular mobilization of LDL through the effects that can be reflected and accentuated by membrane permeability or cholesterol solubilization. Improve the secondary deficiency of sphingomyelinase activity that can be present in these fibroblasts as a manifestation induced by excessive LDL which is accumulated in cell lysosomes, reduces cholesterol accumulation in vascular and extravascular tissues, and partially prevents the development of atherosclerosis induced by the cholesterol from diet (unless by severe hypercholesterolemia accompanying consumed cholesterol), when administered in drinking water to rabbits; reduces bonding, internalization and degradation of exogenous LDL in fibroblasts in cultures of human skin; not acting by the increased cholesterol secretion by the cells.

A study published in 2007 showed a new extraction method of intracellular triglycerides directly through dimethylsulfoxide without use of colorant. The results presented show that DMSO was more effective in extracting intracellular triglycerides than isopropanol (traditionally used solvent) and report that DMSO can dissolve and extract triglycerides directly from cells and the concentrations of triglycerides can be subsequently quantified with a commercial kit for analysis.

DMSO was selected based on the facts: 1) the ability to cross membranes, verified by numerous researchers. 2) DMSO is a versatile and powerful solvent that dissolves a large amount of organic compounds and polymers, including triglycerides. 3) DMSO is a nonvolatile solvent that assures a stable concentration of triglycerides before quantification. 4) Although it is an organic solvent, DMSO did not inhibit the activity of the analysis kit.

The effects of dimethylsulfoxide on accumulation of cyclic AMP, lipolysis and glucose metabolism in fat cells indicate that the DMSO in high concentrations inhibited the cyclic AMP phosphodiesterase and it stimulates the accumulation of cyclic AMP and lipolysis in fat cells.

Dimethylsulfoxide (DMSO) decreased the oxidation of the glucose by stimulated insulin and increased a lipolysis. DMSO also potentialized the increase of lipolysis due to glucagon, norepinephrine or theophylline. The increase in cyclic AMP levels due to these lipolytic agents was by 1.10 M DMSO. This effect was quick in the beginning, since the increase of cyclic AMP due to DMSO was detected 40 seconds after the addition of the lipolytic agents. The decreased accumulation of cyclic AMP seen after the addition of propanolol in fat cells incubated with norepinephrine was reduced by DMSO. DMSO inhibited the activity of cyclic AMP phosphodiesterase both soluble and particulate present in fat cells. DMSO metabolites, dymethylsulphona and dimethyl sulfide, did not cause any changes to accumulation of cyclic AMP due to norepinephrine, showing that the DMSO and not a metabolite was responsible for the effects observed. As mentioned above, this data shows that DMSO in high concentrations inhibited the cyclic AMP phosphodiesterase and stimulates the accumulation of cyclic AMP and the lipolysis in fat cells.

Note: The phosphodiesterase is an enzyme that degrades AMPc (cyclic adenosine monophosphate) which is a type of cell “messenger”. Phosphodiesterase decreases the AMPc levels (since it is degrading it) and these low levels hinder the lipolysis in fat cells, i.e., hinders the break of fat from cells storing it for reservation purposes. A phosphodiesterase inhibitor causes the AMPc to increase and the lipolysis to be more effective.

A summarized comparison of how the mechanism through which DMSO cures cellulite during the physiopathological process of its formation is below.

There is more retention of fluids between blood (capillary) vessels and tissues, where the nutrient exchanges occur. An edematous infiltration, of irregular consistency and changes to membrane permeability hinders the metabolic exchanges. Therefore, there is retention of organic waste and toxin accumulation; since DMSO has a high diuretic effect, even when topically delivered, it avoids hydric retention and hinders nutrient exchange between capillary veins and tissues. DMSO increases membrane permeability, improving metabolic exchanges and balancing cell homeostasis, producing a normalizing effect in the whole organism. Besides, it is a potent antioxidant and anti-free radicals, sequestering the toxins present in cellulite tissue, where the fat is rancid and poorly fed.

There is an increased concentration of fat cells in the area. At this moment the undulations and the “orange peel skin” appearance are already visible; as seen above, DMSO has affinity with fat cells, not destroying other cells. With its enormous capacity of attraction by adipocytes and cross cellular membranes, DMSO as correctly used (and as described hereinafter) will destroy the integrity of these membranes and consequently the adipocyte cell itself, thus eliminating the excessive concentration of fat cells and avoiding the concentration of others.

Due to excessive adipocytes, along with water, toxins, lack of oxygen and nutrients, this rancid fat smashes and “suffocates” vessels and tissues, causing a venous-lymphatic congestion and hyperplasia of local connective tissue fibers, promoting a fibroesclerous process with onset of fat nodules and the “orange peel skin” appearance, with hindered circulation due to microvaricose veins. Constant heaviness, tingling and swelling of legs, besides tiredness, fatigue and pain can also arise; besides being capable of destroying fat cells, increase cell permeability by balancing metabolic exchanges, has a potent diuretic, antioxidant, and anti-free radicals effect, DMSO also has vasodilating action, improving circulation, which promotes cellulite elimination and improves the symptoms of tingling, heaviness and swelling of legs, tiredness, fatigue, and pain. It was also noticed that, as mentioned above, DMSO can be intended to treat subjects with escleroderma, since it can dissolve pathologic deposits of collagen without changing collagen metabolism in normal tissues and without hindering the elastic tissue. Thus, the problem of the awful fat nodules and “orange peel skin” appearance, formed by fat cells and hyperplasia of the connective tissue fibers, is solved.

Note: it can be noticed that cellulite also affects thin women. This occurs because, besides hormonal factors, poor diet and sedentary habits, there is hydric retention, poor oxygenation, poor blood circulation, excessive free radicals and presence of rancid fat. Thus, DMSO will also be effective the whole populations, since obesity is becoming an epidemic in several countries of the world.

Some examples of concentrations used are listed below.

| | |
|---|---|
| DMSO 6% to 99.99% | Aqueous base |
| DMSO 6% to 99.99% | Gel base |
| DMSO 6% to 99.99% | Lotion base |
| DMSO 6% to 99.99% | Aerosol |
| DMSO 6% to 99.99% | Other non-oily bases |
| DMSO 1% to 5% | Aqueous base |
| DMSO 1% to 5% | Gel base |
| DMSO 1% to 5% | Lotion base |
| DMSO 1% to 5% | Aerosol |
| DMSO 1% to 5% | Other non-oily bases |

Other topical concentrations, in form of ointments, gel cream and others, ranging from 1% to 99.99%.
Injectable concentrations ranging from 1% to 99.99% DMSO.
Oral concentrations ranging from 1% to 99.99% DMSO.

Used as described above, dimethyl sulfoxide will promote gradual and progressive elimination of a small portion of adipocytes per each application. As mentioned before, the amount of adipocytes to be destroyed per session/application with DMSO is approximately 50,000-500,000 cells, having a safety and tolerability profile.

Proposed concentrations can also be used to actuate in conjunction with other treatment techniques for cellulite, flabbiness and localized fat, especially in thighs, in more advanced stages; for reduction of the pain, edema and the inflammatory process of cellulite.

The preparation of finished DMSO-based products from 1% to 99.99% comprises the steps of weighing DMSO, completing with the base and homogenizing it.

Study of the Efficacy and Safety of Destroying Adipocytes (Fat Cells) and Eliminating Cellulite and Adiposities.

Cellulite is a disturbance of the subcutaneous layer that adversely changes the overlying superficial appearance of the. Alterations to fibrotic septa between fat cells and tissue reduce metabolic rates, thus congesting the tissue and impacting the external appearance of the skin. The rugosities caused by these alterations can be seen as uneven dimples in the skin. Cellulite occurs from puberty on, in almost every woman, and when it becomes excessive, is hard to control and treat. There are some treatment modalities, but there are few hat really shows a significant improvement and none, until the present time, was capable of eliminating cellulite.

This study was exclusively designed to determine the efficacy and safety of DMSO delivered in several manners and in different concentrations for eliminating cellulite and adiposities.

This is a multicentric, prospective, longitudinal study, where clinical and instrumental parameters are used to detect results regarding different formulations and application forms performed. It is not possible to perform a double-blind study with placebo due to the strong odor of DMSO present in formulations, easy to identify.

A group of 145 subjects, with ages ranging from 18 to 55 years (average 36.5 y.o.), having cellulite for at least 2 years, were recruited from April, 2008 until July, 2008.

Inclusion criteria were: presence of cellulite (stages 1, 2, 3, and 4) in subjects of groups A and B; accentuated abdominal fat in the subjects of the group C; age ranging from 18 to 55 years old and interest in entering the study.

Exclusion criteria were: after liposculpture, other treatments anti-cellulite ongoing or 30 days before entering the study, pregnant and nursing women, cancer, people with hormonal alterations, endocrinological disorders, and people under hormonal or endocrinal treatment.

145 subjects were randomized in 3 groups and received 3 different treatments according to established method. Group A entered 29 female subjects having Dermal Paniculopathy (cellulite) stages 1 and 2, Group B entered 58 female subjects with Dermal Paniculopathy (cellulite) stages 3 and 4, and Group C entered 58 subjects, 35 female and 23 male, all having pronounced abdominal fat.

The three products were formulated as follows: group A consisted in an aqueous solution of DMSO at 20% and aerosol spray to 5%, group B consisted in an aqueous solution of DMSO at 50% and aerosol spray 5%, and group C consisted in an aqueous solution of DMSO at 90% and aerosol spray at 5%.

The aqueous solution was used in the massage applied before the delivery of spray. Each massage session lasted 45 minutes in average.

After massage, the spray was applied at 30 cm (11.80 in) of distance for approximately 5 seconds in each site.

After the procedure as above, a drainage massage was applied for 20 minutes.

In total, each session last 1 hour and 30 minutes in average.

Two sessions were applied per week during 5 weeks.

All subjects were analyzed at the end of each session, and especially after the 10 sessions.

Evaluation methods were: digital photographs (before and after), clinical observation, measurement of thigh circumference, pinching test (for pain), measurement of fat with plicometer/adipometer, measurement of abdominal circumference, measurement of hips and waist circumference, and questionnaire with questions about swelling, pain and heaviness in the legs, cellulite appearance, weight, general efficacy of the product, tolerability, satisfaction with the treatment, sensation of wellbeing, increased diuresis, urine color, among others.

From a total 145 entered subjects, 96% (139) completed the study. Six subjects left the study for personal reasons. After the 10 treatment sessions, the 139 subjects were examined and showed the following statistically significant results.

Group A presenting well-being:

| | |
|---|---|
| Excellent | 20% |
| Great | 44% |
| Good | 28% |
| Regular | 3% |
| Bad | 5% |

General feeling of improvement

| | |
|---|---|
| Excellent | 78% |
| Great | 12% |
| Good | 10% |
| Regular | 0% |
| Bad | 0% |

Improvement from “orange peel skin“ appearance

| | |
|---|---|
| Excellent | 76% |
| Great | 13% |
| Good | 11% |
| Regular | 0% |
| Bad | 0% |

Abdominal circumference reduction

| | |
|---|---|
| Up to 3 cm | 32% |
| 3.1-5 cm | 39% |
| 5.1 cm and more | 29% |

Hip circumference reduction

| | |
|---|---|
| Up to 2.5 cm | 31% |
| 2.6-4.5 cm | 43% |
| 4.5 cm and more | 26% |

Thigh circumference reduction

| | |
|---|---|
| Up to 2 cm | 28% |
| 2.1-4 cm | 48% |
| 4.1 cm and more | 24% |

Weight loss *

| | |
|---|---|
| Up to 2 kg | 31% |
| 2.1-3 kg | 44% |
| 3 kg and more | 25% |

Diuresis improvement

| | |
|---|---|
| Excellent | 28% |
| Great | 39% |
| Good | 25% |
| Regular | 8% |
| Bad | 0% |

Urine color and odor in early stages of the treatment

| | |
|---|---|
| Weak | 32% |
| Regular | 24% |
| Strong | 44% |

Reduced pain in cellulite sites

| | |
|---|---|
| Excellent | 21% |
| Great | 27% |
| Good | 39% |
| Regular | 13% |
| Bad | 0% |

Mood improvement

| | |
|---|---|
| Excellent | 26% |
| Great | 41% |
| Good | 33% |
| Regular | 0% |
| Bad | 0% |

General efficacy

| | |
|---|---|
| Excellent | 74% |
| Great | 17% |
| Good | 9% |
| Regular | 0% |
| Bad | 0% |

General satisfaction after all 10 sessions

| | |
|---|---|
| Excellent | 72% |
| Great | 22% |
| Good | 6% |
| Regular | 0% |
| Bad | 0% |

* All subjects were told to keep their regular habits, without changing diet and physical activities.

From 139 subjects, 30% had accentuated problems of venous insufficiency and varicose veins.

Such problems entail pain, peso, swelling, tingling legs, arms, and hands. There were the following reports:

| | |
|---|---|
| Decreased pain in legs, arms and hands | 62% |
| Decreased tingling legs and arms | 55% |
| Decreased swelling and heaviness in the legs | 73% |
| Decreased and whitened macrovaricose veins | 39% |

Topical delivery of DMSO showed an excellent safety and tolerability profile. Adverse reactions commonly reported in a period of 2 hours right after the application were: nausea (27 subjects or 19%), headache (15 subjects or 11%), feeling of burn in application site (11 subjects or 8%), itch and redness in application site (14 subjects or 10%), hypotension (7 subjects or 5%), odor and taste similar to garlic or corn (136 subjects or 98%), dry mouth (32 subjects or 23%), increased diuresis (98 subjects or 70%), strong urine color and odor (68 subjects or 49%). These reactions were evaluated as light and transitory.

Increased diuresis with strong urine color and odor were also reported during the days following the application, as well as an odor similar to garlic and corn. A subject reported the increased blood pressure 2 weeks after starting the treatment; however, this was reported as non-related to DMSO, since the patient was concomitantly taking allopathic drugs for other pathologies. No patient discontinued the treatment due to adverse reactions.

An improvement of all clinical signs associated to cellulite (edemas in legs, heaviness, pain, “orange peel skin” appearance, etc.) was in fact observed by both subjects receiving DMSO applications and the professionals accompanying the treatment and evaluating the results.

The balanced diminishment of all measured circumferences and a statistically significant diminishment of weight loss indicate the elimination of the adipocytes present in cellulite and fat tissues.

The sensation of wellbeing, improved mood, increased diuresis, improved aspect of the skin and satisfaction in general showed the benefic systemic effects of DMSO after topical application, besides presenting an excellent safety and tolerability profile.

The data obtained showed that the DMSO investigated can eliminate the adipocytes present in cellulite tissue, besides actuating in several physiopathological stages involved in cellulite pathogenesis and, thus, is safe and effective in improving all signals and symptoms associated thereto.

The good results obtained in this study also suggest good adherence from the subjects, with sessions regularly repeated and standardized application performed to all subjects.

Considering all the findings, the pharmacokinetics, high permeability, all characteristic properties and the action mechanism in which DMSO actuates in eliminating cellulite and its “orange peel skin” appearance and adiposity (localized fat), there is the possibility of the results obtained lasting longer than the period of time evaluated in this study. It is necessary, however, maintenance program with regular intervals and more investigations.

Many products are commercially available for treating adiposities. The biggest function is reducing linear body measures, something demanding much more treatment complexity.

One can mention a wide range of products intended for fighting cellulite and adiposities, among those: artichoke polyphenols; lipostabil (phosphatidylcholine); nordihydroguaiaretic acid (NDGA); caffeine; centella asiatica; seaweed; ivy; lipolytic salt; Equisetum ssp.; Solanum cernuum; Aesculus Hippocastanum; Ilex paraguariensis; methyl nicotinate; magnesia sulphate; thiomucase; cellulinol; ginkgo biloba; mallow; ginseng; salicylic acid.

The literature of Dimethyl Sulfoxide (attached) shows that it has numerous indications; however, in more than 30,000 scientific journals researched from all over the world, there is no mention of its action in eliminating cellulite and adiposity, neither about its use in destroying adipocytes.

### DETAILED DESCRIPTION OF THE INVENTION

Thus, the present patent application “USE OF DMSO FOR DESTROYING ADIPOCYTES AND ELIMINATING CELLULITE AND ADIPOSITIES” shows a product which, until the present time, did not exist in consumer's market at all, i.e., no drug, cosmetic, clinical or even surgical procedure available capable of destroying and consequently diminish the amount of fat cells excessively deposited in the organism.

By destroying an approximate amount from fifty thousand (50,000) to five hundred thousand (500,000) adipocytes (fat cells) per application of DMSO, cellulite and adiposities are gradually destroyed in its primary causes and, consequently, by the end of some sessions, we will obtain the definitive solution (elimination) of cellulite and adiposities (localized fats). The vasodilating, antioxidant and free radical sequestering effects of DMSO also have an important role for eliminating the “orange peek skin” appearance of cellulite since the latter shows, as mentioned above, as rancid, poorly oxygenated, and poorly fed fat with accumulation of toxins.

The main objective of the present invention relates to the use of DMSO (dimethyl Sulfoxide) for destroying adipocytes, eliminating cellulite and adiposities.

The concentrations proposed herein can also be used to actuate in connection with other treatment techniques for cellulite and localized fat.

Thus, we hope to help people spending along their lives a significant amount of money in the desperate search for the cure or at least improvement of these ills and mostly not obtaining the success or satisfaction they hope for.

It is evident, however, that practice of physical activities, good diet, regular intake of pure water, healthy habits and keeping your mind with uplifting thoughts and constructive feelings are vital for a balanced, ruled life, and for a healthy and nice organism.

The present invention refers to the use of DMSO (Dimethyl Sulfoxide) with the objective of destroying adipocytes (fat cells), thus eliminating the “orange peel skin” appearance of cellulite, as well as adiposities for both males and females.

Previous patent application, PI 0504655-6 of Oct., 17^{th}, 2005, filed by the same Applicant, referred to the use of DMSO in fighting cellulite and other adiposities.

However, the main characteristic of the present invention is the fact that, according to the results of a new research, it was observed that DMSO, instead of only dissolving and removing the fat present inside the adipocytes and eliminating it main through urine, which actually happens is the break or destruction of membranes and, consequently and completely, the adipocyte cells.

This is particularly important, since when fat cells only wither or diminish in size, as occurs during weight loss, there is a large risk of the fat returns to the initial site (inside cells) as soon as one starts eating fats again and keeping bad habits.

As described above, the cells are kept in place, although smaller, there is an improvement in the “orange peel skin” appearance mainly caused by the presence of adipocytes, but not the elimination.

By destroying adipocytes (e.g., as in liposuction procedures) the “orange peel skin” appearance is also eliminated when combined with other DMSO actions against further factors contributing to the formation of cellulite, as described hereinafter.

Since cellulite is mainly characterized by the localized presence of rancid, poorly fed, and poorly oxygenated fat and with accumulation of toxins, instead of only treating cellulite, it will eliminate it, i.e., with maximum advance. in this case it is not only a palliative, but a definitive solution.

It is right, when it comes to destruction of the adipocytes present in cellulite, it is also considered the use of DMSO for eliminating other adiposities and/or localized fats.

Briefly, when destroying adipocytes (fat cells) there is the elimination of fat cells, which is fraught with rancidness and toxins. The rancid fat cell causes cellulite, since the cell maintains the cellulite nodule. The cellulite nodule in its turn is shown as the “orange peel skin” appearance, which is more common in women and easily identified according to the stage of cellulite. In the meantime, by eliminating the fat cell, the adiposity is naturally reduced, and as consequence the localized fat is reduced.

Even though the invention is descripted herein in detail, it is important to understand that its application is not limited to the details and steps described herein. The invention is capable of other modalities and being practiced or executed in various forms. It must be understood that the terminology applied herein is for purposes of description, not limitation.

1. “USE OF DMSO FQR DESTROYING ADIPOCYTES AND ELIMINATING CELLULITE AND ADIPOSITIES”, obtained from usual concentration of DMSO, used for palliative treatment of cellulite and other adiposities, wherein DMSO concentrations a re used to completely and totally eliminate cellulite and adiposities are presented as follows:

| | | |
|---|---|---|
| DMSO - | 6% to 99.99% | in Aqueous base |
| DMSO - | 6% to 99.99% | in Gel base |
| DMSO - | 6% to 99.99% | in Lotion base |
| DMSO - | 6% to 99.99% | in Aerosol |
| DMSO - | 6% to 99.99% | in Other non-oily bases |
| DMSO - | 1% to 5% | in Aqueous base |
| DMSO - | 1% to 5% | in Gel base |
| DMSO - | 1% to 5% | in Lotion base |
| DMSO - | 1% to 5% | in Aerosol |
| DMSO - | 1% to 5% | in Other non-oily bases |

2. “USE OF DMSO FOR DESTROYING ADIPOCYTES AND ELIMINATING CELLULITE AND ADIPOSITIES” as claimed in claim 1, wherein topical concentrations as ointments, gel cream and others, range from 1% to 99.99%.

3. “USE OF DMSO FOR DESTROYING ADIPOCYTES AND ELIMINATING CELLULITE AND ADIPOSITIES”, as claimed in claim 1, wherein injectable concentrations range from 1% to 99.99% DMSO.

4. “USE OF DMSO FOR DESTROYING ADIPOCYTES AND ELIMINATING CELLULITE AND ADIPOSITIES”, as claimed in claim 1, wherein oral concentrations range from 1% to 99.99% DMSO.

5. “USE OF DMSO FOR DESTROYING ADIPOCYTES AND ELIMINATING CELLULITE AND ADIPOSITIES”, as claimed in claim 1, wherein the amount of adipocytes to be destroyed in each session/delivery of DMSO is approximately 50,000-500,000 cells.

6. “USE OF DMSO FOR DESTROYING ADIPOCYTES AND ELIMINATING CELLULITE AND ADIPOSITIES”, as claimed in claim 1, wherein the formulations are used to act in conjunction with other treatment techniques for cellulite, flabbiness and localized fat, especially on the fat of the thighs in more advanced stages; to reduce pain, edema and cellulite inflammatory process.

7. “USE OF DMSO FOR DESTROYING ADIPOCYTES AND ELIMINATING CELLULITE AND ADIPOSITIES”, as claimed in claim 1, wherein the preparation of DMSO concentrations ranging from 1% to 99.99% comprises the steps of: weighing DMSO, supplementing with the base and homogenizing.

8. “USE OF DMSO FOR DESTROYING ADIPOCYTES AND ELIMINATING CELLULITE AND ADIPOSITIES”, as claimed in claim 1, wherein the concentrations are used to act in conjunction with other treatment techniques for cellulite and localized fat.

9. “USE OF DMSO FOR DESTROYING ADIPOCYTES AND ELIMINATING CELLULITE AND ADIPOSITIES”, as claimed in claim 1, wherein DMSO presents the following structure: [chemical structure]
